(19) United States
(12) Patent Application Publication
(10) Pub. No.: US 2010/0196195 A1
(43) Pub. Date: Aug. 5, 2010

(54) AUTOMATIC AIR FRESHENER

(76) Inventor: Tim Moschel, West Harrison, IN (US)

Correspondence Address: DINSMORE & SHOHL LLP, FIFTH THIRD CENTER, ONE SOUTH MAIN STREET, SUITE 1300, DAYTON, OH, 45402-2023, US

(21) Appl. No.: 12/696,460

(22) Filed: Jan. 29, 2010

Related U.S. Application Data
(60) Provisional application No. 61/149,091, filed on Feb. 2, 2009.

(51) Int. Cl. A61L 9/00; A61L 9/14; A61L 9/04; F24F 3/12; B01F 3/04

(52) U.S. Cl. 422/4; 422/124; 422/116; 422/5; 261/72.1

(57) ABSTRACT

A liquid delivery device for use in conjunction with an air filter in a forced air circulation system. The device includes a liquid reservoir, a dispersion device connected to the reservoir, a compressor, and a mounting device configured to slidably retain an air filter. The device may be used in conjunction with an HVAC system.

This application claims the benefit of U.S. Provisional Application Ser. No. 61/149,091 (DRF 0001 MA), filed Feb. 2, 2009.

### BACKGROUND OF THE INVENTION

Forced air systems have been disclosed that include features to allow the addition of a liquid to an air circulation system. Such systems, however, require modification to the air filter in order to add fragrance to the circulating air. Each also requires wicking material to be inserted within the filter media, which requires specialized filter media, different what is commonly provided on the marketplace. The modification adds expense and difficulty in finding a suitable replacement when the old filter must be replaced.

In other configurations, the amount of fragrance released is evenly distributed throughout the useful life of the filter. Unfortunately, this approach can lead to over-saturation of the air supply when too much fragrance is dispensed and unnoticeable levels, such as when not enough of the fragrance is released. Furthermore, these devices are configured such that the units cannot be operated unless the device is oriented vertically, on a stable platform. What is needed is a device that overcomes these difficulties.

### SUMMARY

The present invention relates generally to a liquid delivery device for use in conjunction with a forced air heating and cooling system. Although the liquid delivery device and methods of the present disclosure are not limited to particular configurations or the context in which the device is being used, for the purposes of illustration, the device and method steps are illustrated herein with reference to specific assemblies.

It is contemplated that the processes of the present disclosure will also enjoy utility in other air circulation devices, including those that are structurally similar to or distinct from the devices illustrated herein.

In accordance with one embodiment of the present disclosure, a liquid delivery device for use in conjunction with an air filter in a forced air circulation system is discussed. The device comprises a liquid reservoir configured to contain a liquid, a dispersion device fluidly connected to the liquid reservoir, a compressor cooperative with the liquid reservoir to facilitate delivery of the liquid to the dispersion device, and a mounting device attached to the liquid delivery device configured to retain an air filter.

Optionally, device may include a number of potential variations. The dispersion device may comprise an evaporation pad or spray nozzle. The liquid delivery device may also include an electronic controller to operate a compressor. Furthermore, the electronic controller may include a timer electrically connected to a compressor. Optionally, the electronic controller may also include a differential pressure switch. In one aspect, the air filter and liquid reservoir is optionally detachable. In another optional configuration, the mounting device comprises at least one channel configured to slidably retain standard forced air circulation systems filters.

In accordance with another embodiment of the present disclosure, a method of dispersing a liquid into a forced air circulation system is discussed. The method comprises providing a liquid delivery device comprising a liquid reservoir containing a liquid, and a compressor cooperative with said liquid reservoir and a dispersion device responsive to said liquid reservoir. The method also comprises pressurizing the liquid reservoir with the compressor to provide liquid to the dispersion device, and dispersing the liquid into a circulating air stream with the dispersion device.

Optionally, dispersing the liquid may including spraying the liquid into a circulating air stream. Alternatively, dispersing the liquid may include contacting an evaporation pad 16A with the liquid and contacting the evaporation pad 16A with circulating air. Furthermore, it is contemplated that the liquid is selected from group including deodorants, bactericides, decongestants, inhalants, aromatics, fragrances, insecticides, repellants, medicants, and pharmaceuticals. In one optional configuration, the mounting device is configured to slidably retain standard forced air circulation systems filters.

In another optional configuration, the pressurizing step may be conducted in timed cycles. Alternatively, the pressurizing step may have a duration inversely proportional to the amount of cycles completed for a volume of liquid. Furthermore, the dispersing step may optionally include ramping the amount of liquid released in each cycle.

In accordance with another embodiment of the present disclosure, a heating, ventilation, and air-conditioning system with a liquid delivery device is discussed. The HVAC system comprising a forced air circulation system, at least one air delivery duct connected to the forced air circulation system, and an air filter fluidly connected to the forced air circulation system. The liquid delivery device comprises a liquid reservoir configured to contain a fluid, a dispersion device fluidly connected to the liquid reservoir, a compressor cooperative with liquid reservoir to facilitate delivery of the liquid to the dispersion device, and a mounting device configured to slidably retain the air filter.

Optionally, the system may also include an electronic controller which selectively operates the liquid delivery device. The electronic controller may also include a timer connected to the compressor configured to disperse the liquid in timed cycles.

### BRIEF DESCRIPTION OF THE SEVERAL VIEWS OF THE DRAWINGS

The following detailed description of specific embodiments of the present disclosure can be best understood when read in conjunction with the following drawings, where like structure is indicated with like reference numerals and in which:

FIG. 1 shows a front view of a device for use in conjunction with a forced air circulation system for the present invention accordance with one embodiment;

FIG. 2 shows a front perspective view of a device for use in conjunction with a forced air circulation system for the present invention in accordance with another embodiment;

FIG. 3 shows a front view of a device for use in conjunction with a forced air circulation system in accordance with yet another embodiment;

FIG. 4 shows a front perspective view of a detached liquid reservoir usable with the devices of FIGS. 1 and 2;

FIG. 5 shows a side view of an HVAC system with the liquid delivery device of FIGS. 1 and 2.

### DETAILED DESCRIPTION

In one embodiment, a liquid delivery device 1 for use in conjunction with a forced air circulation system is provided. It is contemplated that the liquid delivery device 1 may be compatible with a range of HVAC and other forced air circulation systems, including, but not limited to residential and commercial furnaces, air conditioners, climate control devices, fans, and vents. However, the liquid delivery device 1 may also be modified for use in other systems involving a circulating air stream. The forced air circulation system contemplated for use in conjunction with the liquid delivery device 1 employs an air filter 5 to capture dirt, dust, or debris from the circulating air stream.

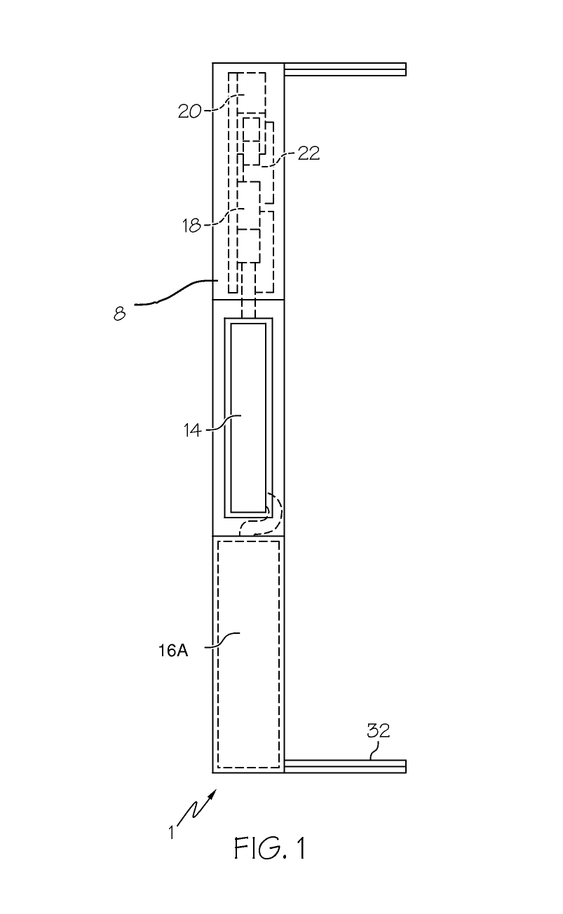

Referring to FIG. 1, in one embodiment, a liquid delivery device 1 for use in conjunction with an air filter 5 in a forced air circulation system. The liquid delivery device 1 may comprise a liquid reservoir 14 configured to contain a liquid, and a dispersion device 16 fluidly connected to the liquid reservoir 14. The liquid delivery device 1 may also comprise a compressor 18 cooperative with the liquid reservoir 14 to facilitate delivery to a dispersion device, and a mounting device configured to retain a conventional air filter 5. In the version shown, the mounting device is shaped as one or more channels that can slidably accept the edge of the air filter 5. It will be appreciated that those skilled in the art that other fastening or related connection approaches (not shown) may be employed and that such approaches are within the scope of the present invention.

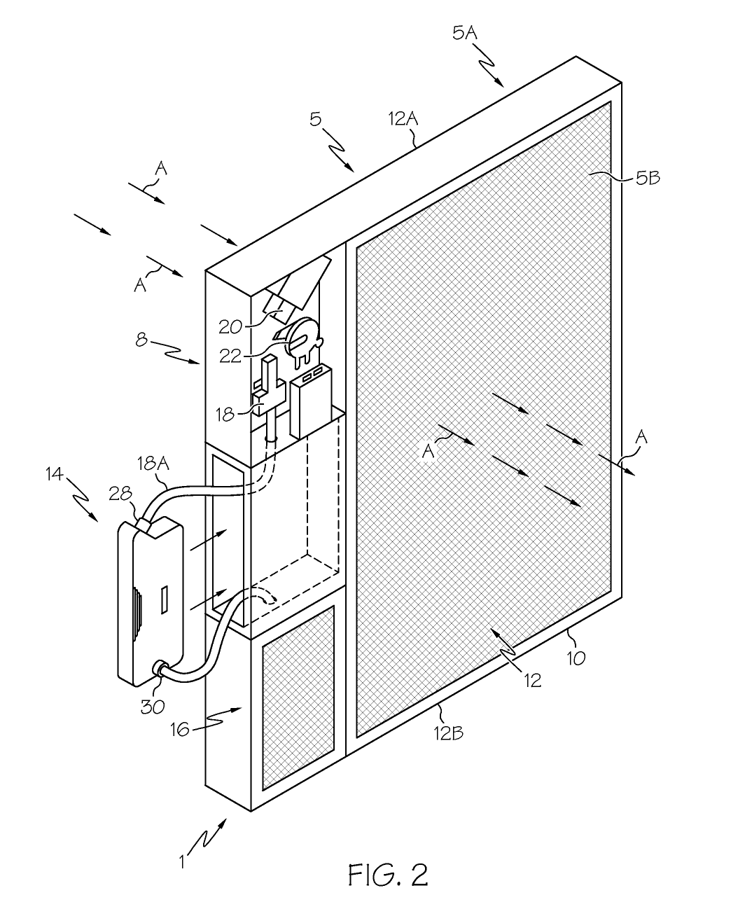

Referring to FIG. 2, the air filter 5 defines an inlet side 5A and an outlet side 5B, that corresponds to an airflow direction A. In addition, the air filter 5 may include a filter media 12 disposed fluidly between the inlet and outlet side. It is contemplated that the air filter 5, or more particularly, the filter media 12 may take a variety of shapes and formats to ensure compatibility with a range of forced air systems. For example, the air filter 5 may be rectangular, round, square, or some other more specialized shape. The air filter 5 may also comprise a range of sizes and thicknesses. For example, the width of the air filter 5 may range from about 8 to about 30″, and the height may range from about 8 to about 40″. Preferably, the air filter 5 is dimensionally sized to be interchangeable with standard forced air circulation system filters. By standard, it is understood that the filter configuration is compatible with commonly available air filter sizes and configurations commonly available.

In one aspect, the air filter 5 defines an inlet and outlet side that corresponds to the inlet side 5A and outlet side 5B of the air filter 5, with a filter media 12 disposed within the frame 10. Typically, the filter media 12 will have one side configured to accept incoming circulating air, known as the inlet side 12A. Furthermore, the filter media 12 may have one side configured to discharge passing through circulating air, known as the outlet side 12B.

The filter media 12 may comprise a range of materials. For example, glass fibers, polypropylene fibers, PVC fibers, and polyamide fibers can be used. More generally, polyacrylonitrile and polyvinyladine chlorides can be used. Other suitable synthetic polymeric fibers can be used to make filters including polysulfone, sulfonated polysulfone, polyvinylidine fluoride, polyvinyl chloride, chlorinated polyvinyl chloride, polycarbonate, nylon, aromatic nylons, cellulose esters, aerolate, polystyrene, polyvinyl butyryl, and copolymers of these various polymers.

Referring to FIGS. 1 & 2, in one embodiment, the liquid delivery device 1 may comprise a liquid reservoir 14, a dispersion device 16 fluidly connected to the liquid reservoir 14, and a compressor 18 cooperative with the liquid reservoir 14. The liquid delivery device 1 may be contained within a housing 8. However, it is also contemplated that the liquid delivery device 1 and air filter 5 are co-mingled within a frame 10. Alternatively, the frame 10 may comprise four sections, thereby allowing the mechanical and electrical devices to work with the forced air circulation system while still functioning independently.

Referring to FIG. 4, the liquid reservoir 14 may comprise a detachable tank that may be refilled or replaced. Alternatively, the liquid reservoir 14 may be permanently affixed to the housing 8, and connected to the compressor 18, such that the liquid reservoir 14 is non-detachable and non-refillable, and such that it is replaceable as part of the liquid delivery device 1. Preferably, the liquid reservoir 14 comprises a sealing device that allows the liquid reservoir 14 to seal upon connection to the liquid delivery device 1. Upon insertion into the liquid delivery device 1, the liquid reservoir 14 will be fluidly connected to the compressor 18 and dispersion device 16.

The liquid reservoir 14 may comprise a variety of different materials suitable to contain a liquid. Materials may include, but are not limited to, polymers, plastics, fiberglass, or other synthetic or natural materials. The liquid reservoir 14 may comprise a variety of shapes and forms, operable to contain a liquid and fluidly connect to a dispersion device 16. In one configuration, the liquid reservoir 14 may have a stand pipe connecting the liquid reservoir 14 to the dispersion device 16. Preferably, a tube will be provided within the liquid reservoir 14 from the top of the liquid reservoir 14 to the bottom, except for a small gap. This configuration allows the liquid delivery device 1 to function in different orientations, whether vertical or horizontal.

In another embodiment, the liquid reservoir 14 has a nozzle mounted on the bottom side of the reservoir 14. The nozzle is fluidly connected to the dispersion device 16 to provide liquid in either a horizontal or vertical orientation. Pressure provided to the top of the liquid reservoir 14 may force liquid through the nozzle and to the dispersion device 16. Alternatively, it is contemplated that liquid may be transferred from the liquid reservoir 14 to the dispersion device 16 with other components suitable for multiple orientation operation.

In another embodiment, the liquid reservoir 14 comprises a check valve 28 operable to retain liquid when the liquid reservoir 14 is oriented such that liquid contacts the check valve 28. The check valve 28 is also to operable to retain pressure accumulated in the liquid reservoir 14 through operation of a compressor 18. The check valve 28 may be located within the connection between the compressor 18 and the liquid reservoir 14 such that additional pressure may added to the liquid reservoir 14 without allowing leakage of the liquid.

The liquid reservoir 14 may also comprise an outlet valve 30 disposed between the liquid reservoir 14 and the dispersion device 16 operable to allow liquid to be transferred to the dispersion device 16 when a predetermined pressure has been reached. The threshold amount of pressure may be adjusted in order to accommodate a range of possible dispersion devices as discussed below. Additionally, the threshold pressure may vary to accommodate different forced air circulation system capacities. The outlet valve 30 may have a number of possible configurations operable to retain the liquid until a threshold pressure is met.

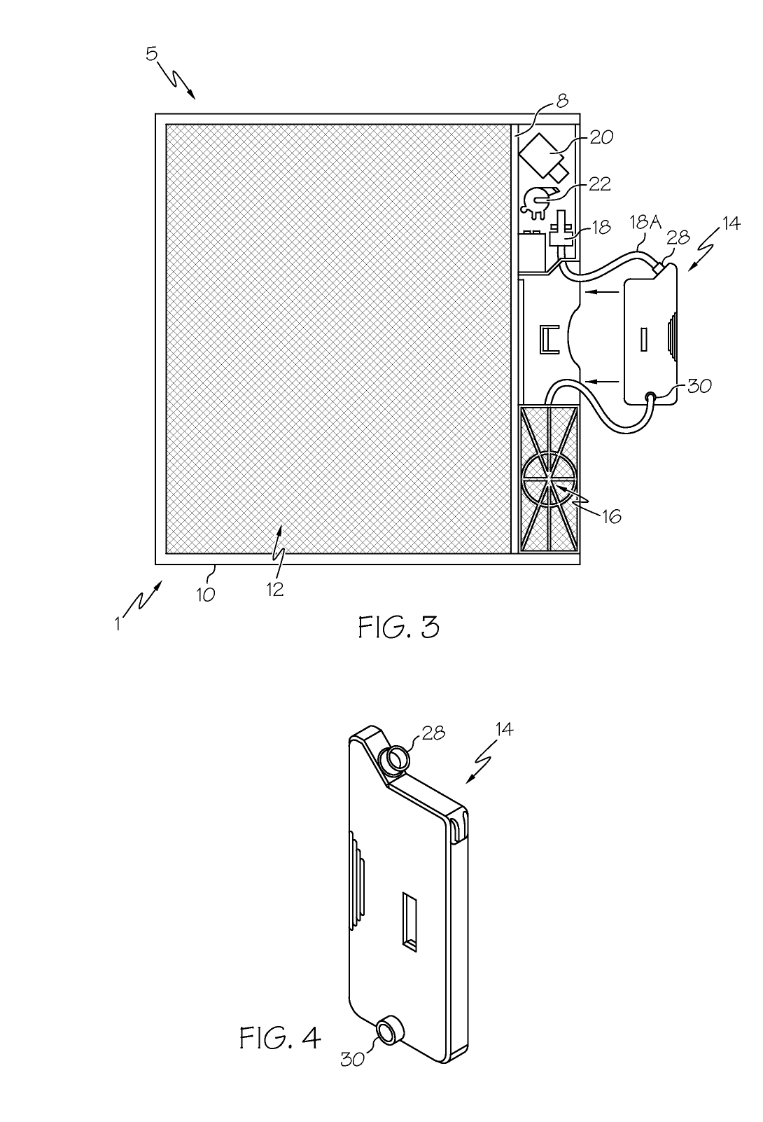

Referring to FIG. 3, the liquid delivery device 1 may comprise a compressor 18 cooperative with the liquid reservoir 14 via connection tube 18A to facilitate the delivery of liquid to the dispersion device 16. Preferably, the compressor 18 pressurizes the liquid reservoir 14 by filling it with air. The compressor 18 may comprise a variety of configurations, suitable to pressurize the liquid reservoir 14. For example, the compressor 18 may comprise a pump. Alternatively, the compressor 18 may comprise other devices suitable to provide pressure to the liquid reservoir 14, such as a fan, motor, or other similar device. In one embodiment, the compressor 18 transfers air into a liquid reservoir 14 until a predetermined pressure is reached depending on the level of liquid remaining in the liquid reservoir 14.

In one aspect, pressure at the top of the liquid will force the liquid from the liquid reservoir 14 up the tube, and out of the end of the tube to the dispersion device 16. Preferably, when the liquid is forced up the tube in the liquid reservoir 14, it will proceed by gravity to the dispersion device 16. In another aspect, the liquid is transferred from the liquid reservoir 14 to a dispersion device 16 through other means suitable to transfer pressurized liquid.

In one embodiment, the dispersion device 16 may comprise an evaporation pad 16A. Upon transfer to the evaporation pad 16A, the liquid is contacted with circulating air to cause evaporation into the forced air circulation system. The evaporation pad 16A may comprise a range of materials, suitable to absorb the fluid, and subsequently allow for evaporation to the circulating air. Suitable materials may include, but are not limited to natural materials, man-made materials, fibrous materials, non-fibrous materials, porous materials, non-porous materials, and combinations thereof. Preferably, the evaporation pad 16A does not have a density greater than common filter material used in residential forced air circulation systems. In another aspect, the evaporation pad 16A may serve as a collection point so liquid may not drip into the filter frame area. In another embodiment, the dispersion device 16 may comprise a spray nozzle (not shown). Liquid may be transferred to the spray nozzle for distribution into the circulating air stream. The spray nozzle may comprise a range of devices, including but not limited to an aperture, an atomizer, or other similar spraying device. However, it is also contemplated that the dispersion device 16 may also be other devices suitable to infuse the liquid into a circulating air stream.

Referring further to FIG. 1, in one embodiment, the mounting device may comprise at least one channel 32 configured to slidably retain a standard forced air circulation systems filter. The size of the at least one channel 32 is configured to allow a filter 5 to slide into the channel 32 into a position adjacent to the liquid delivery device 1. The channel 32 is sized to allow the filter to slide and be frictionally retained. The channels 32 may extend laterally from a housing 8 disposed around the liquid delivery device 1. Alternatively, a single channel 32 may be provided to allow the slidably retain an air filter 5. Alternatively, the at least one channel 32 may take other shapes and orientations suitable to retain an air filter. The channels 32 may be of any length operable to slidably retain an air filter 5. For example, the channels 32 may extend from a housing 8 ranging from 0.5 cm to 30 cm, or 2 to 20 cm, or other lengths. Generally, the channel 32 may comprise metal, cardboard, plastic, or other suitable material.

Referring to FIG. 2, in another embodiment, the mounting device may comprise a frame 10 that allows an air filter 5 to be slidably retained. The frame 10 may be configured to allow an air filter 5 to be slid within one side of the frame 10 and retained adjacent to the liquid delivery device 1. Upon exhaustion of the useful life of the air filter 5, it may be slid out of the frame 10, and replaced. The frame 10 may comprises a variety of different shapes and configurations. Generally, the frame 10 may made of metal, cardboard, plastic, or other material.

In one embodiment, the liquid delivery device 1 also comprises an electronic controller 20 configured to selectively operate the device 1 and dispense a controlled amount of liquid. Preferably, the electronic controller 20 controls whether the compressor 18 pressurizes the liquid reservoir 14. The electronic controller 20 may be cooperative with or responsive to a sensor that signals taken from the sensor can be used as feedback for the electronic controller 20. The electronic controller 20 may comprise a microprocessor-based electronic control system that provides functions interactive with the forced air circulation system. In one configuration, the electronic controller 20 has a ramping function preprogrammed to start delivering liquid at a lower level, increase the amount of dispersed liquid to a peak level, and then begin diminishing the amount dispersed to a lower level. Preferably, the ramping function creates a perceptive effect in liquid vaporized that is not normally provided with even, or continuous distribution. Perceptive effect is hereby understood to mean the impact that the dispersed liquid has on a person within an effective range of the liquid delivery device 1. In one aspect, the ramping function is controlled by the amount of pressure introduced in the liquid reservoir 14 by the compressor 18. In another configuration, as the liquid level in the liquid reservoir 14 changes, the timer corresponds with the electronic controller 20 to pressurize the liquid reservoir 14 for an appropriate amount of time. However, other controlling systems are also contemplated.

Since the liquid level may change, and the pressurizing step may be constant, there is a variable amount of liquid dispersed, which contributes to perceivable changes in the air circulation area. In another aspect, the electronic controller 20 may be programmed to control the amount of liquid dispersed during each pressurizing cycle. For example, the electronic controller 20 may be programmed to have its first five cycles to be operational for a period ranging from about 2 seconds to about 3 seconds, the second set of five cycles to run for about 3 to about 5 seconds. In addition to cycle time and pressure, the amount of time that the compressor 18 runs can be varied to deliver a preferred amount of fluid.

In one embodiment, the electronic controller 20 cooperates with the compressor 18 to control the pressurizing step. Preferably, the pressurizing step is conducted in discrete cycles, rather than in a continuous, or semi-continuous process. However, it is also contemplated that the compressor 18 is run continuously or semi-continuously.

In another aspect, the pressurizing step has a duration inversely proportional to the amount of cycles completed for a given volume of fluid. Preferably, the compressor 18 runs for a longer period of time after successive cycle. Accordingly, the liquid reservoir 14 may be maintained at a substantially similar pressure during each successive dispersion step. As liquid is dispersed to the circulating air, the amount of liquid contained in the liquid reservoir 14 diminishes. Preferably, the compressor 18 may run for a sufficient length of time or at a sufficient capacity to reach a desired operating pressure within the liquid reservoir 14. In one embodiment, the operating pressure in the liquid reservoir 14 may range from about 0.001 and about 2 PSI. Alternatively, the device may be configured to operate at other pressure levels.

In yet another embodiment, the electronic controller 20 comprises a differential pressure switch 22 that is functionally connected to the inlet and outlet sides of the air filter 5 to detect a pressure change and provide power to the compressor 18 and electronic controller 20. Alternatively, power may directly supplied to the electronic controller 20 and compressor 18. Preferably, the differential pressure switch 22 is configured to detect a pressure differential between the inlet side and outlet side of the air filter 5. The switch 22 is typically activated by fan operation in the forced air circulation system. The difference in pressure allows a set of contacts to close and allows power to be passed through the contacts energizing mechanical devices, such as the compressor 18 and the electronic controller 20. In one aspect, the electronic controller 20 and compressor 18 are simultaneously energized when the pressure differential switch 22 closes. In addition, other types of switches may be utilized in conjunction with the compressor 18 and electronic controller 20. For example, manual, temperature, timed, programmable, and other switch types are contemplated.

Alternatively, if the fan on the HVAC system is operated in the manual setting such that the pressure differential switch is activated, the electronic controller 20 is configured to track the dispensing time cycle as long as the differential pressure switch is engaged. Accordingly, the electronic controller 20 may be configured to limit the amount the dispersion time. The electronic controller 20 may sense whether the HVAC system is in manual mode, and switch the timer function automatically. The timer may continue to run during the manual mode, and communicate the interval time to the electronic controller to ensure that an appropriate amount of liquid is dispersed.

In another embodiment, upon actuation of the pressure differential switch 22, the compressor 18 may begin to run, and the electronic controller 20 may begin a timing function, controlling the run-time of the compressor 18. When a predetermined time is met, the electronic controller 20 disconnects the compressor 18 from a power supply. The timer may continue to count, and reengage the compressor 18 after a specified length of time has elapsed, and ultimately reactivate the compressor 18 to start another cycle. In one aspect, if the blower on a forced air circulation system starts, and continues to run for a one hour period, the processor will restart the compressor 18 at predetermined time intervals. In another aspect, if the blower is set to a manual position, which creates a constant pressure differential across the air filter 5, the compressor 18 may be controlled by a timing function independent of the pressure differential switch 22. It is also contemplated that in between each pressurizing cycle, the compressor 18 may activated to provide a baseline resting pressure in the intermittent period during pressurizing and dispersing steps.

If a predetermined pressure differential is detected, the pressure differential switch 22 is activated, and power may be supplied to the electronic controller 20, and ultimately, the compressor 18 and timer. The predetermined pressure differential is preferably based on the particular forced air circulation system utilized, and calibrated such that the pressure differential switch 22 is activated when the circulation system is in active mode.

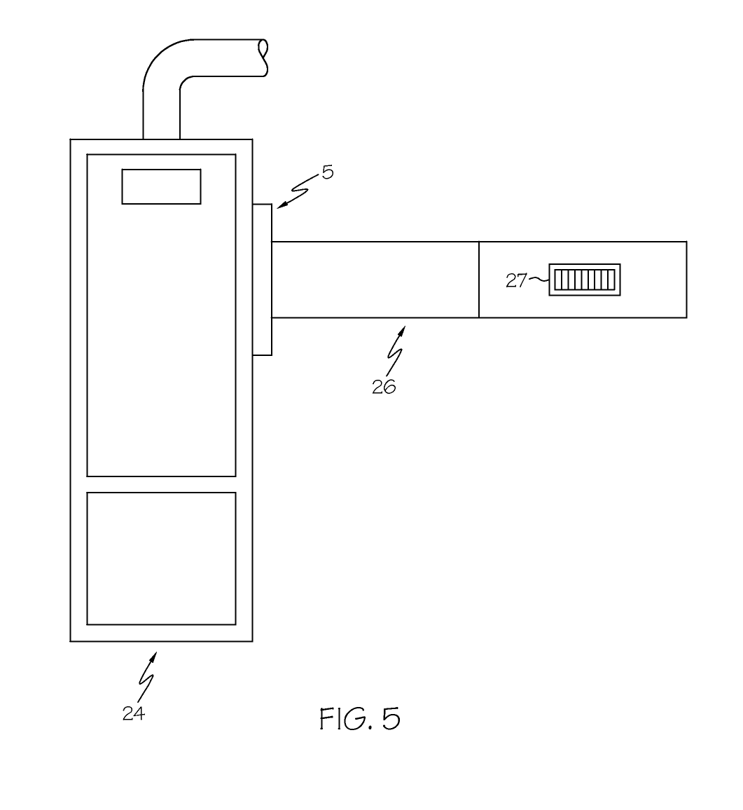

Referring to FIG. 5, an HVAC system 24 with a liquid delivery device 1 is provided. At least one air delivery duct 26 and register or vent 27 is connected to the HVAC system 24, with an air filter 5 comprising a liquid delivery device 1. It is contemplated that the HVAC system 24 may comprise a variety of forms and configurations, such as typical residential and commercial systems, as well as other more specialized systems. Generally, at least one air delivery duct 26 is connected to the HVAC system 24 which enables efficient transport of air. However, an HVAC system without ducts is also contemplated for use in conjunction with the liquid delivery device 1, utilizing other means of transporting air.

Preferably, the liquid provided herein is a material that is vaporizable. As such, a volatile material can comprise an “aroma” and a “scent” including, but not limited to, pleasant or savory smells, materials having antibacterial, antiviral, and insecticidal functions. For example, volatile materials may include insecticides, additives, air fresheners, deodorants, aromacology, aromatherapy, or any other odor that acts to condition, modify, or otherwise charge the atmosphere or to modify the environment. However, other liquids are also contemplated for use in the present invention.

For the purposes of describing and defining the present invention, it is noted that reference herein to a variable being a “function” of a parameter or another variable is not intended to denote that the variable is exclusively a function of the listed parameter or variable. Rather, reference herein to a variable that is a “function” of a listed parameter is intended to be open ended such that the variable may be a function of a single parameter or a plurality of parameters.

It is also noted that recitations herein of “at least one” component, element, or the like, should not be used to create an inference that the alternative use of the articles “a” or “an” should be limited to a single component, or element.

It is noted that recitations herein of a component of the present disclosure being “programmed” in a particular way, “configured” or “programmed” to embody a particular property, or function in a particular manner, are structural recitations, as opposed to recitations of intended use. More specifically, the references herein to the manner in which a component is “programmed” or “configured” denotes an existing physical condition of the component and, as such, is to be taken as a definite recitation of the structural characteristics of the component.

It is noted that terms like “preferably,” “commonly,” and “typically,” when utilized herein, are not utilized to limit the scope of the claimed invention or to imply that certain features are critical, essential, or even important to the structure or function of the claimed invention. Rather, these terms are merely intended to identify particular aspects of an embodiment of the present disclosure or to emphasize alternative or additional features that may or may not be utilized in a particular embodiment of the present disclosure.

For the purposes of describing and defining the present invention it is noted that the terms “substantially” and “approximately” are utilized herein to represent the inherent degree of uncertainty that may be attributed to any quantitative comparison, value, measurement, or other representation. The terms “substantially” and “approximately” are also utilized herein to represent the degree by which a quantitative representation may vary from a stated reference without resulting in a change in the basic function of the subject matter at issue.

Having described the subject matter of the present disclosure in detail and by reference to specific embodiments thereof, it is noted that the various details disclosed herein should not be taken to imply that these details relate to elements that are essential components of the various embodiments described herein, even in cases where a particular element is illustrated in each of the drawings that accompany the present description. Rather, the claims appended hereto should be taken as the sole representation of the breadth of the present disclosure and the corresponding scope of the various inventions described herein. Further, it will be apparent that modifications and variations are possible without departing from the scope of the invention defined in the appended claims. More specifically, although some aspects of the present disclosure are identified herein as preferred or particularly advantageous, it is contemplated that the present disclosure is not necessarily limited to these aspects.

What is claimed is:

1. A liquid delivery device for use in conjunction with an air filter in a forced air circulation system, said device comprising:
a liquid reservoir;
a dispersion device fluidly connected to said liquid reservoir;
a compressor cooperative with said liquid reservoir to facilitate delivery of a liquid contained therein to said dispersion device; and
a mounting device attached to said liquid delivery device to retain an air filter.

2. The device of claim 1, wherein said dispersion device comprises an evaporation pad.

3. The device of claim 1, wherein said dispersion device comprises a spray nozzle.

4. The device of claim 1, further comprising an electronic controller configured to selectively operative said compressor.

5. The device of claim 4, wherein said electronic controller comprises a timer electrically connected to said compressor operable to pressurize said liquid reservoir for varying amounts of time.

6. The device of claim 4, wherein said electronic controller further comprises a differential pressure switch connected to an inlet side and an outlet side of said air filter.

7. The device of claim 1, wherein said mounting device comprises at least one channel configured to slidably retain at least one standard forced air circulation systems filter therein.

8. The device of claim 1, wherein said mounting device comprises a frame, wherein said frame is dimensionally sized to slidably retain a standard forced air circulation systems filter.

9. The device of claim 1, wherein said liquid reservoir is detachable from said liquid delivery device.

10. A method of dispersing a liquid into a forced air circulation system, said method comprising:
providing a liquid delivery device comprising a liquid reservoir containing a liquid, a compressor cooperative with said liquid reservoir and a dispersion device responsive to said liquid reservoir;
pressurizing said liquid reservoir to provide liquid to said dispersion device; and
dispersing said liquid into a circulating air stream with said dispersion device.

11. The method of claim 10, wherein dispersing said liquid comprises spraying said liquid into said circulating air stream.

12. The method of claim 10, wherein dispersing said liquid comprises contacting an evaporation pad with said liquid and contacting said evaporation pad with said circulating air.

13. The method of claim 10, wherein said liquid is selected from group consisting of deodorants, bactericides, decongestants, inhalants, aromatics, fragrances, insecticides, repellants, medicants, and pharmaceuticals.

14. The method of claim 10, wherein the liquid is a fragrance.

15. The method of claim 10, wherein said pressurizing step is conducted in timed cycles.

16. The method of claim 15, wherein said pressurizing step has a duration inversely proportional to the amount of cycles completed for a volume of liquid.

17. The method of claim 15, wherein said dispersing step further comprises ramping the amount of liquid released each cycle to achieve maximum perceptive effect over time.

18. A heating, ventilation, and cooling system with a liquid delivery device, the system comprising:
a forced air circulation system;
at least one air delivery duct connected to said forced air circulation system;
an air filter fluidly connected to said forced air circulation system; and
a liquid delivery device cooperative with at least one of said air filter and said air delivery duct, comprising:
a liquid reservoir configured to contain a liquid;
a dispersion device fluidly connected to said liquid reservoir;
a compressor cooperative with said liquid reservoir to facilitate delivery of said liquid to said dispersion device;
a mounting device configured to slidably retain said air filter.

19. The system of claim 18, further comprising an electronic controller configured to selectively operate said liquid delivery device.

20. The system of claim 18, wherein said electronic controller further comprises a timer functionally connected to said compressor configured to disperse the liquid in timed cycles.
